(19) United States
(12) Patent Application Publication
(10) Pub. No.: US 2017/0347769 A1
(43) Pub. Date: Dec. 7, 2017

(54) NAIL CLEANER

(71) Applicants: Edward SAPIENZA, New York, NY (US); Norman TURKOWITZ, New York, NY (US)

(72) Inventors: Edward SAPIENZA, New York, NY (US); Norman TURKOWITZ, New York, NY (US)

(21) Appl. No.: 15/451,000

(22) Filed: Mar. 6, 2017

Related U.S. Application Data
(60) Provisional application No. 62/304,264, filed on Mar. 6, 2016.

(51) Int. Cl. A45D 29/17; A45D 29/04; A61B 17/54; B08B 1/00

(52) U.S. Cl. CPC A45D 29/17; B08B 1/005; A45D 29/04; A61B 17/54

(57) ABSTRACT

A compact nail cleaner device made of an elongated portion 4 attached to a circular disc component 3 having an inner cavity 1 and an outermost surface with ridges 2

This application claims the benefits of U.S. Provisional application Ser. No. 62/304,264 filed Mar. 6, 2016, which is incorporated herein in its entirety by reference.

### FIELD OF THE INVENTION

The present invention relates to a nail cleaner. More particularly, the invention relates to a nail cleaning device that quickly and easily removes dirt and debris from underneath a person's fingernails and/or toenails.

### BACKGROUND OF THE INVENTION

There are a variety of nail cleaners on the market and each product has its own drawbacks. For example, for a nail brush to effectively clean underneath the fingernail, water must be used.

Representative prior art includes U.S. Pat. No. 4,119,439 To Boucher which discloses an apparatus to clean underneath a person's fingernails. The apparatus is used in beauty salons and industries were hand cleanliness is required. The person's fingers are held in the apparatus and a stream of water, detergent and antiseptic solution is delivered through orifices therein to clean the crevices between the person's fingers and their nails to dislodge and dissolve dirt.

U.S. Pat. No. 4,289,152 to Fuhre also discloses a finger nail cleaning apparatus. The user inserts the fingertip to be cleaned and the apparatus separates the fingertip from the finger nail for cleaning.

U.S. Pat. No. 5,640,979 to Trenary discloses a finger nail cleaner apparatus with a rotating brush assembly.

Other representative prior art includes U.S. Pat. No. 8,640,715 to Howell which discloses an emery board for cleaning nails but has no ridges along the side.

U.S. Pat. No. 4,886,078 to Shiffman discloses an elongated rod or stick with one end pointed and tufted to engage under the finger or toe nails for cleaning. The rod or stick is made of a porous material that can be impregnated with cleaning agents.

U.S. Pat. No. 5,090,427 to Sherts disclose a finger groomer attachment to a writing device. The finger nail cleaner has a pointed edge for cleaning underneath the nails.

U.S. Pat. No. 5,652,988 to Appelhoff and U.S. Pat. No. 5,706,835 to Salvino both disclose handheld devices for cleaning and manicuring fingernails.

U.S. Patent Publication No. US 2009/0090377 to Tran discloses a multi-function fingernail device having a brush on one end and a scrapper or a second brush attached to the other end.

Other products located on the internet at www.gojo.com illustrate disposable flat plastic nail cleaners with a pointed end to clean out dirt and debris from underneath the user's fingernail.

However, none of the prior art references disclose a nail cleaning device made of a elongated portion with a circular disc portion perpendicular to the elongated portion which serves as a handle for the user. The circular disc portion has ridges on the outer most surface which are parallel to the handle. These ridges are used to clean underneath the nail surface, while the opposite end can be round or pointed and used to clean other parts of the nail as well as underneath the nail surface.

None of the prior art references discloses the invention alternate embodiment which in addition to the circular disc includes an elongated portion made of an emery board material with ridges along its side. In both invention embodiments the structure of the device enables the user to clean hard to reach places like underneath the user's nail

The invention overcomes the disadvantages of the prior art by providing a compact pocket size device that consists of an elongated portion connected to circular disc having ridges to easily and quickly clean under the nail surface. The elongated portion is for gripping the device and may include ridges on the side for additional cleaning and grooming.

The circular disc has ridges along the outermost surface and an inner cavity. This inner cavity of the circular disc easily fits on the tip of a person's fingernail for easy manipulation to clean underneath the finger or toe nails of the person. The circular disc is positioned underneath the nail to be cleaned and is then rotated back and forth by the individual. This motion quickly and easily removes dirt and debris from under the nail without the need for water or other cleaners. In the embodiment where there is a pointed tip on the elongated portion, this is tip is also used to help remove dirt and debris from the nails. Thus, the entire device is used to easily clean an individual's nails without any additional materials or mess.

The invention nail cleaner provides a compact pocket size nail cleaner device which can easily be used by both adults and children.

An advantage of the present invention is in the provision of a small pocket size nail cleaner that is compact and inexpensive.

Another advantage of the invention is that the nail cleaner is disposable and in some embodiments usable, depending on the material it's made of.

### SUMMARY OF THE INVENTION

In the present invention, these purposes, as well as others which will be apparent, are achieved generally by providing a one piece nail cleaner device made of an elongated portion and a circular disc component located perpendicular to the elongated portion. The circular disc has ridges on the outermost surface which run parallel to the elongated portion. The circular disc has an inner cavity which easily fits on a person's finger for easy nail cleaning.

The elongated portion is used for easy gripping of the device and is preferably flat. The elongated portion may further include ridges on the sides for additional cleaning or may have a textured surface which may be used to file nails or rough skin.

Other objects, features and advantages of the present invention will be apparent when the detailed description of the preferred embodiments of the invention are considered in reference to the drawings, which should be construed in an illustrative and not limiting sense.

### BRIEF DESCRIPTION OF THE DRAWINGS

FIG. 1 is an illustration of the nail cleaner and components according to the invention and includes an upside down view displaying the pointed tip end 5 and a right side up view displaying the circular disc component 3 with ridges 2 including the inner cavity 1;

FIG. 2 is another illustration of the nail cleaner and components according to the invention showing an upside down view including dimensions of the tip width a, the circular disc component width b, the angle of the circular disc component c and the distance between the circular disc component and the elongated body d;

FIG. 3 is another illustration of the nail cleaner and components according to the invention showing a cross section side view including dimensions of the tip point e and the length of the elongated body f;

FIG. 4 is an illustration of the top view of the circular disc component showing the inner cavity 1 along with the diameter of the inner cavity g and the width of the circular disc component including ridges h; and an enlargement (Scale 8:1) of the ridges of the circular disc component A (shown in FIG. 4A) along with angle between the ridges i and the dimensions of each ridge j, k; as well as a cross section B-B (shown in FIG. 4B) along with the depth of the inner cavity I;

FIG. 5 is an illustration of the invention in an upside down position with the pointed tip end upwards;

FIG. 6 is an illustration of the invention in a side profile position with the circular disc portion on top showing the inner cavity 1; and

FIG. 7 includes illustrations described in FIGS. 7A to 7G and show an alternate embodiment of the elongated portion 4 of the invention, including dimensions.

### DETAILED DESCRIPTION OF THE INVENTION

As used in this specification and drawings the components of the invention are described numerically as follows:

1—inner cavity of the circular disc component;

2—ridges along the outermost surface of the circular disc component;

3—circular disc component;

4—elongated portion;

5—pointed tip end; and

6—central point within inner cavity.

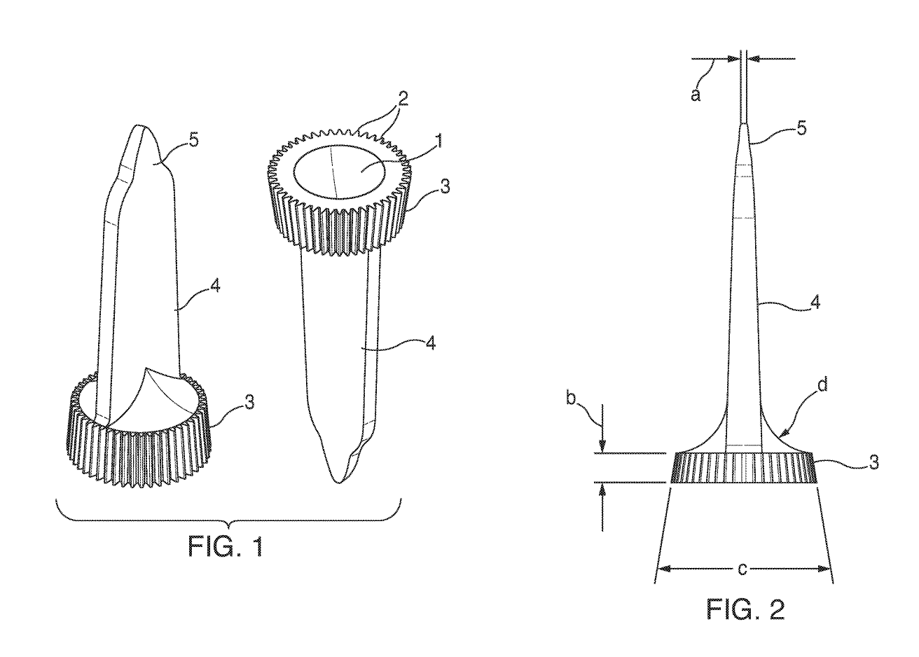

As shown in FIG. 1 the invention provides a compact pocket size nail cleaner device that consists of an elongated portion 4 with a pointed end 5 and on the opposite end a perpendicular circular disc component 3. The elongated portion is for gripping the device and may include ridges on the side. FIG. 1 includes an upside down view displaying the pointed tip end 5 and a right side up view displaying the circular disc component 3 with ridges 2 including the inner cavity 1.

The circular disc component 3 has ridges 2 along the outermost surface and an inner cavity 1. This inner cavity 1 of the circular disc 3 easily fits on the tip of a person's fingernail for easy manipulation to clean underneath the finger or toe nails of the person.

To clean the user's nail, the circular disc 3 is positioned underneath the nail to be cleaned and is then rotated back and forth by the individual. This motion quickly and easily removes dirt and debris from under the nail without the need for water or other cleaners. The pointed tip 5 of the elongated portion 4 is also used to help remove dirt and debris from the nails. Thus, both ends of the device are used to clean an individual's nails without any mess or additional materials.

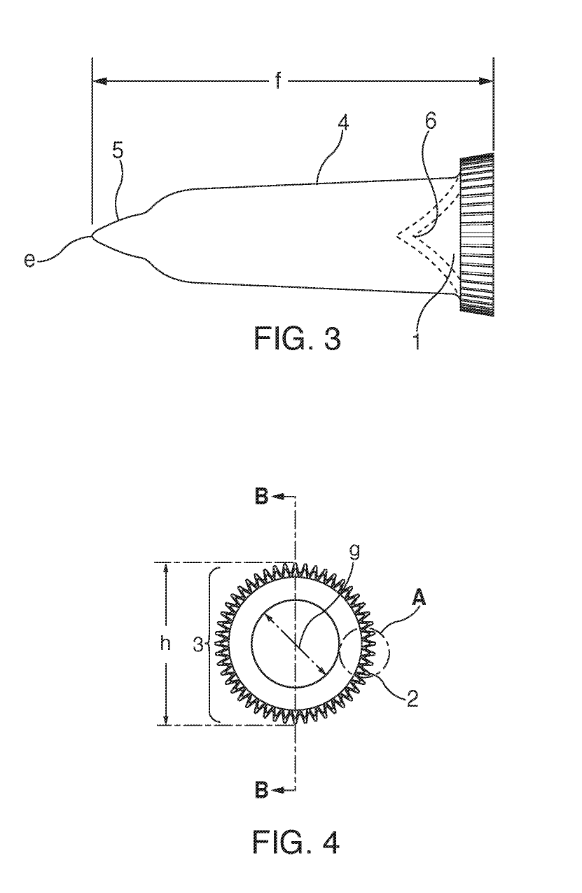

FIG. 2 is another illustration of the nail cleaner and components according to the invention showing an upside down view. Dimensions of the tip width a are in the range of 0.2 to 0.4 inches, preferably 0.3 inches.

The circular disc component has a width b in the range of 0.3 to 1 inch, preferably 0.5 inches and is generally universal for all users but can be customized to the desired width for a users finger which can vary depending on whether it is an adult or child or man or woman.

The angle of the circular disc component c in FIG. 2 is preferably 20° but can range between 30° to 40°.

The circular disc component and the elongated portion are an integral piece. The distance between the circular disc component and the elongated body d is preferably 0.25 inches but can range between 0.1 and 0.3 inches.

FIG. 3 is another illustration of the nail cleaner and components according to the invention showing a cross section side view exposing the inner cavity 1. The dimension of the tip point e is preferably 0.02 inches but can range up to 0.5. The smaller the tip the easier it is for the user to clean hard to reach dirt and debris under the nail. The length of the elongated body f is preferably 1.5 to 1.7 inches but includes embodiments which maybe longer up to 3 or 5 inches.

As seen in FIG. 3, the inner cavity 1 is an open space with the inner surface converging at a central point 6 within the circular disc component 3. This open space creates an almost suction like action when the user places the circular disc component on a nail tip to hold it in place while cleaning.

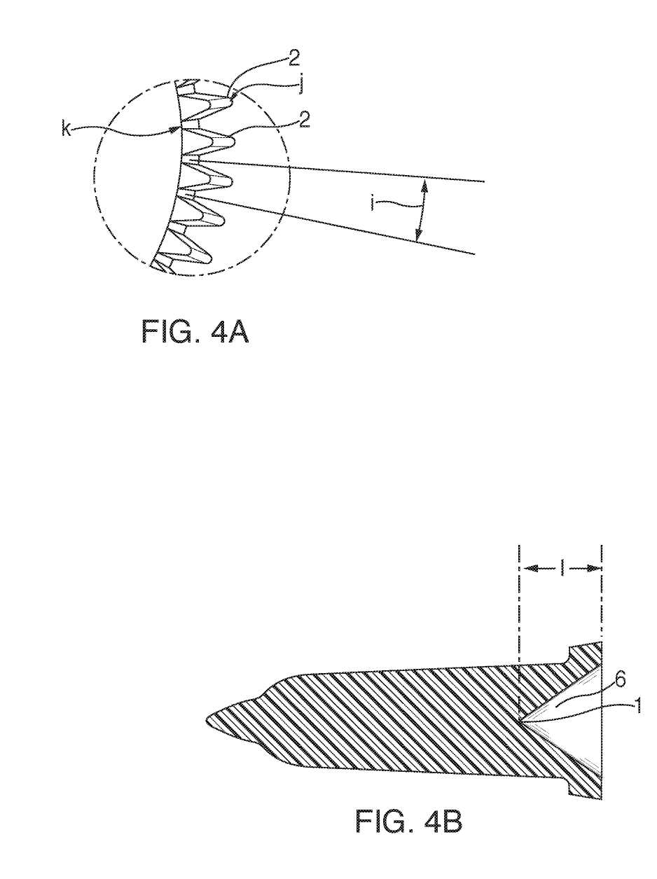

FIG. 4 is an illustration of the top view of the circular disc component 3 showing the inner cavity 1 along with the diameter of the inner cavity g which is 0.3 to 1 inch, preferably 0.5 inches. The width of the circular disc component including ridges h is between 0.4 to 1.1 inches, preferably between 0.6 to 0.7 inches.

FIG. 4 A shows an enlargement (Scale 8:1) of the ridges of the circular disc component A. The angle between each of the ridges i is between 5° to 10° preferably 7.5°. The dimensions of each ridge j, k are between 0.001 to 0.1 inches, preferably 0.005 inches.

As shown in FIG. 4B a cross section B-B from FIG. 4 illustrates a side view of the invention device exposing the inner cavity 1. The depth of the inner cavity I ranges from 0.2 to 0.5 inches, preferably 0.3 to 0.4 inches. The inner cavity is shown as an open space with the inner surface converging at a central point 6 within the circular disc component 3.

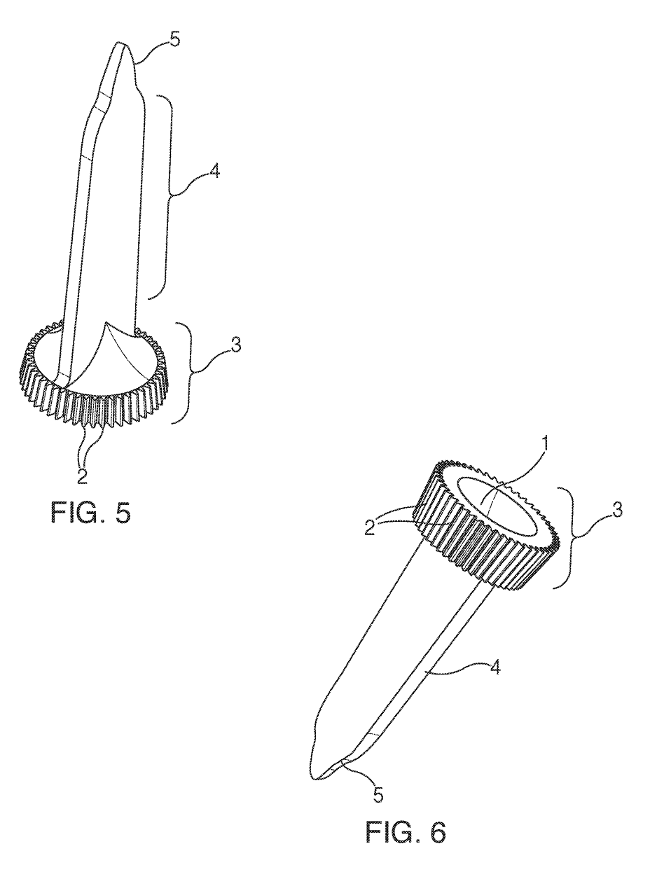

FIG. 5 is an illustration of the invention in an upside down position with the pointed tip end upwards.

FIG. 6 is an illustration of the invention in a side profile position with the circular disc portion on top showing the inner cavity 1.

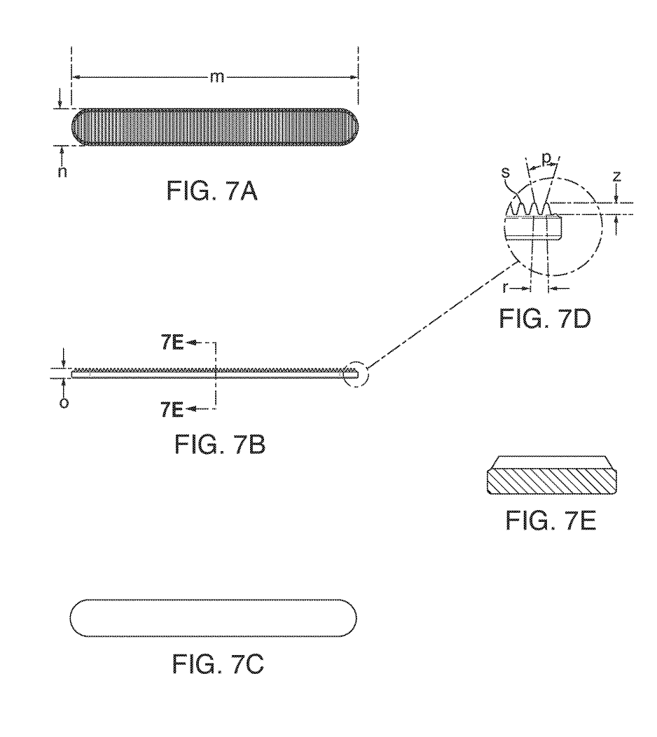

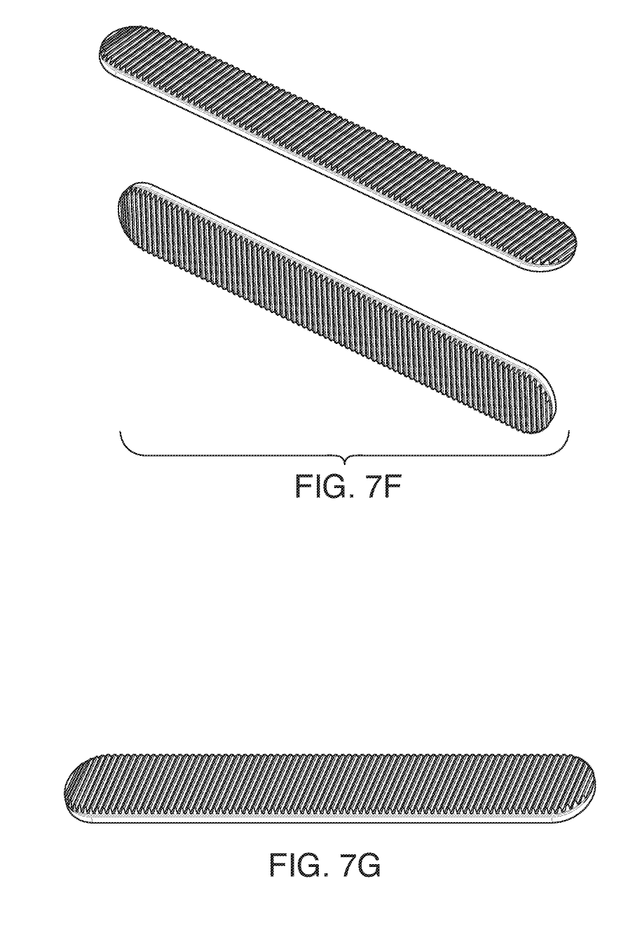

FIG. 7 includes various illustrations FIGS. 7A to 7G. In particular these figures show an alternate embodiment of the elongated portion 4 of the invention, including dimensions. Specifically FIG. 7A illustrates the length and width of the device which includes ridges on the surface for cleaning nails. FIG. 7B shows a side profile of the device and FIG. 7C shows the outer ridge of the device. FIG. 7D is an enlarged scale (8:1) of a portion of the device shown in FIG. 7B.

FIG. 7E is an enlarged scale (8:1) of a portion of the device show in FIG. 7B.

FIG. 7G is an illustration of the textured surface that can be incorporated into the elongated portion 4 of the invention device. For example, instead of a point tip end the elongated portion may include a rounded tip. In addition, the elongated portion may have ridges on at least one side and/or a textured surface on at least one side to further clean dirt and debris and smooth skin and nail surfaces.

The nail cleaner device is made of a material that is hard and durable. The material can be made of plastic, rubber, metal, synthetic materials or any material that can be used to clean nails without breaking.

The invention device can be disposable or reusable depending on the material used.

The device is preferably made by molding in one integral piece. In alternate embodiments the components may be made separately and attached together as one integral piece.

The circular disc has ridges along the outermost surface and an inner cavity. This inner cavity of the circular disc easily fits on the tip of a person's fingernail for easy manipulation to clean underneath the finger or toe nails of the person. The circular disc is positioned underneath the nail to be cleaned and is then rotated back and forth by the individual. This motion quickly and easily removes dirt and debris from under the nail without the need for water or other cleaners. The pointed tip of the elongated portion is also used to help remove dirt and debris from the nails. Thus the device is used to clean an individual's nails without any additional materials or mess.

As described in the previous paragraph the invention also provides a method to clean nails comprising the steps providing a nail cleaner device having an elongated portion 4 attached to a circular disc component 3 which has an inner cavity 1 and an outermost surface with ridges. The user holds the elongated portion 4 in one hand and places the circular disc component 3 under the users nail tip. The circular component 3 is twisted and rotated underneath the nail tip to clean dirt and debris.

In an alternate embodiment a pointed tip end 5 on the elongated portion opposite the circular disc component 3 is provided. The user uses the pointed end to remove further dirt and debris from under the nail tip.

In another embodiment the nail cleaner device elongated portion 4 further includes ridges along at least one side surface such that the user can us the ridges on the elongated portion to remove further dirt and debris from under the nail tip or to smooth skin surfaces.

While dimensions and ranges are stated in the specification herein as preferred ranges, other dimensions are intended to be encompassed by the invention.

The nail cleaner according to the invention comes in various sizes for use by men, women and children.

The foregoing description of various and preferred embodiments of the present invention has been provided for purposes of illustration only, and it is understood that numerous modifications, variations and alterations may be made without departing from the scope and spirit of the invention as set forth in the following claims.

What is claimed is:

1. A nail cleaner device comprised of:
an elongated portion 4 attached to a circular disc component 3 having an inner cavity 1 and an outermost surface with ridges 2.

2. The nail cleaner device according to claim 1, wherein said elongated portion 4 comprises a pointed end 5 on said elongated portion opposite said circular disc component 3.

3. The nail cleaner device according to claim 1, wherein said circular disc component 3 is perpendicular to said elongated portion 4.

4. The nail cleaner device according to claim 1, wherein said ridges 2 are parallel to said elongated portion.

5. The nail cleaner device according to claim 1, wherein said elongated portion 4 is flat.

6. The nail cleaner device according to claim 1, wherein said elongated portion is round.

7. The nail cleaner device according to claim 1, wherein said elongated portion 4 further includes ridges along at least one side surface.

8. The nail cleaner device according to claim 1, wherein said elongated portion has a length in the range of 1 to 5 inches.

9. The nail cleaner device according to claim 2, wherein said pointed end 5 has a width in the range of 0.2 to 0.4 inches.

10. The nail cleaner device according to claim 1, wherein said circular disc component has an inner opening width in the range of 0.3 to 1 inch.

11. The nail cleaner device according to claim 1, wherein said inner cavity 1 depth ranges between 0.2 to 0.5 inches.

12. The nail cleaner device according to claim 1, wherein said inner cavity 1 is an open space with the inner surface converging at a central point 6 within the circular disc component 3.

13. The nail cleaner device according to claim 1, wherein said ridges 2 have a width between 0.001 to 0.1 inches.

14. The nail cleaner device according to claim 1, wherein the angle between said ridges is between 5° to 10°.

15. The nail cleaner device according to claim 1, wherein the device is made of a material selected from the group consisting of plastic, rubber, metal, and synthetic material.

16. The nail cleaner device according to claim 1, wherein the device is one integral piece.

17. The nail cleaner device according to claim 1, wherein the device is compact and portable.

18. A method to clean nails comprising the steps of:
providing a nail cleaner device having an elongated portion 4 attached to a circular disc component 3 which has an inner cavity 1 and an outermost surface with ridges;
wherein said user holds said elongated portion 4 in one hand and places said circular disc component 3 under a users nail tip;
the user twists said circular component 3 underneath the nail tip to clean dirt and debris.

19. The method according to claim 18, wherein the nail cleaner device elongated portion 4 comprises a pointed end 5 on said elongated portion opposite said circular disc component 3; such that the user uses said pointed end to remove further dirt and debris from under the nail tip

20. The method according to claim 18, wherein the nail cleaner device elongated portion 4 further includes ridges along at least one side surface; such that the user uses said ridges on said elongated portion to remove further dirt and debris from under the nail tip.
